(19) United States
(12) Patent Application Publication
(10) Pub. No.: US 2019/0000310 A1
(43) Pub. Date: Jan. 3, 2019

(54) DILATION DEVICES, METHODS, AND SYSTEMS

(71) Applicant: I360MEDICAL LTD., Dublin (IE)

(72) Inventors: Walter PRENDIVILLE, Kilcock (IE); Raymond O'SULLIVAN, Kilkenny (IE); Derek YOUNG, Dublin (IE); Conor HAND, Dublin (IE)

(73) Assignee: i360medical Ltd., Dublin (IE)

(21) Appl. No.: 16/060,871

(22) PCT Filed: Dec. 9, 2016
(86) PCT No.: PCT/IB2016/001752

§ 371 Date: Jun. 8, 2018

Related U.S. Application Data
(60) Provisional application No. 62/265,654, filed on Dec. 10, 2015.

(51) Int. Cl. A61B 1/32; A61M 29/02; A61B 1/00; A61B 1/303; A61M 39/22

(52) U.S. Cl. CPC A61B 1/32; A61M 29/02; A61M 39/22; A61B 1/303; A61B 1/00135

(57) ABSTRACT

A dilation system (10) comprising a speculum (20) with a body and at least one jaw (24,26) pivotally attached to the body; and a sheath (30) that is attached on the body around the at least one jaw, and expandable from a constricted position to an expanded position. A dilation comprising: at least one proximal ring (133P) spaced apart from at least one distal ring (133D); a plurality of longitudinal ribs (32) spanning between each of the proximal and distal rings; and a web (34) spanning between at least each of the plurality of ribs, wherein the sheath is expandable from a constricted position to an expanded position. Related methods are also disclosed.

### CROSS-REFERENCE TO RELATED APPLICATIONS

This application claims the benefit of priority of U.S. Provisional Application No. 62/265,654, filed Jan. 10, 2015, the entirety of which is incorporated by reference into this application.

### BACKGROUND

Numerous medical procedures require dilation of a body lumen, such as those specific to the female reproductive system. A metal speculum is commonly used to perform these procedures. Metal speculums often rely upon mechanical ratchets, or like mechanisms, to maintain the speculum in an expanded position. These mechanisms may not provide the surgeon with adequate control over the amount of dilation, thus causing discomfort or vaginal injury, such as tearing. Metallic speculums can also be cold, adding further discomfort. Further improvements are required.

### SUMMARY

Aspects of the present disclosure relate to dilation devices, methods, and systems. Numerous aspects of the present disclosure are now described with reference to an exemplary dilation device comprising: at least one proximal ring spaced apart from at least one distal ring; a plurality of longitudinal ribs spanning between each of the proximal and distal rings; and a web spanning between at least each of the plurality of ribs, wherein the sheath is expandable from a constricted position to an expanded position. This exemplary device may be used in conjunction with other device described herein, or within any system or method described herein. It may be understood that both the foregoing summary and the following detailed descriptions are exemplary and explanatory only, neither being restrictive of the inventions claimed below.

### BRIEF DESCRIPTION OF THE DRAWINGS

The accompanying drawings, which are incorporated in and constitute a part of this specification, illustrate exemplary aspects that, together with the written descriptions, serve to explain the principles of this disclosure.

FIGS. 1A-B depicts an exemplary dilation device including a body with a movable jaw.

FIG. 2 depicts an exemplary system including the device of FIGS. 1A-B and an exemplary sheath mounted on the device.

FIG. 3 depicts another view of the system of FIG. 2.

FIG. 4A depicts a side view of the system of FIG. 2.

FIG. 4B depicts a top view of the system of FIG. 2.

FIG. 5 depicts a back view of the system of FIG. 2.

FIG. 6A depicts a perspective view of the system of FIG. 2.

FIG. 6B depicts another perspective view of the system of FIG. 2.

FIG. 7 depicts another exemplary sheath.

FIG. 8 depicts yet another exemplary sheath.

### DETAILED DESCRIPTION

The present disclosure is now described with reference to exemplary aspects of dilation devices, methods, and systems. Some aspects are depicted and/or described with reference to a particular body orifice or lumen, such as the vagina, rectum, mouth, or other naturally occurring orifices; while other aspects reference a particular procedure, such as those specific to any of these orifices or lumens. These references are provided for convenience and not intended to limit the present disclosure unless incorporated into the appended claims. Accordingly, the concepts and novelty underlying each aspect may be utilized for any analogous type of lumen or orifice, natural or otherwise; or procedure, medical or otherwise.

The directional terms “proximal” and “distal” are used to describe relative components and features of the present disclosure. The term proximal refers to a position closer to the exterior of the body or a user, whereas the term distal refers to a position closer to the interior of the body or further away from the user. Unless claimed, these directional terms are provided for convenience and not intended to limit the present disclosure to a particular direction or orientation.

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. Unless stated otherwise, the term “exemplary” is used in the sense of “example,” rather than “ideal.”

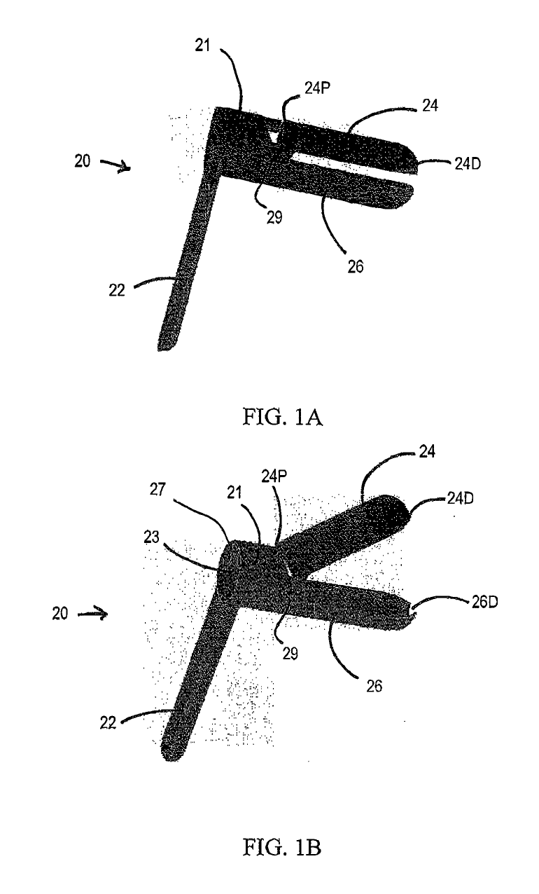

One aspect of the present disclosure is depicted in FIG. 1 as a dilation device illustrated as a speculum 20 including a body 21 and at least one jaw 24 pivotally attached to body 21. Jaw 24 has a distal end 24D opposite of a proximal end 24P, which is pivotable about a pivot point 29 relative to a lower jaw 26 having a distal end 26D. The proximal end of jaw 26 is integral with body 21, but may be pivotally attached thereto. A lumen 23 extends through body 21 from the distal ends 24D and 26D of jaws 24 and 26 to a proximal end 21P of body 21. Lumen 23 is sized to receive another medical device therein, such as an elongated instrument. The respective distal ends 24D and 26D of jaws 24 and 26 may have a semi-circular opening sized to receive or secure a portion of said elongated instrument therebetween. Although not always required, a handle 22 extends away from body 21 to provide a grip for speculum 20. Handle 22 may be angled relative to body 21 so as to achieve discrete, comfortable, and ergonomic use of speculum 20 for both patient and medical practitioner. Speculum 20 also has a port 27 on the proximal end of body 21. Port 27 may, for example, provide a path for guiding the elongated instrument into a body lumen. For example, port 27 may be used to guide a camera or light source through lumen 23 of body 21 and into a vagina for visual inspection of a cervix.

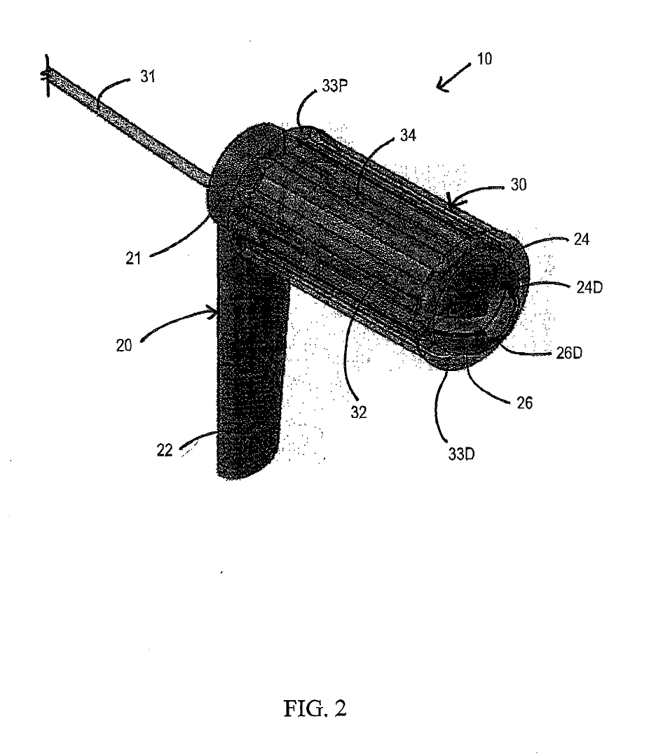

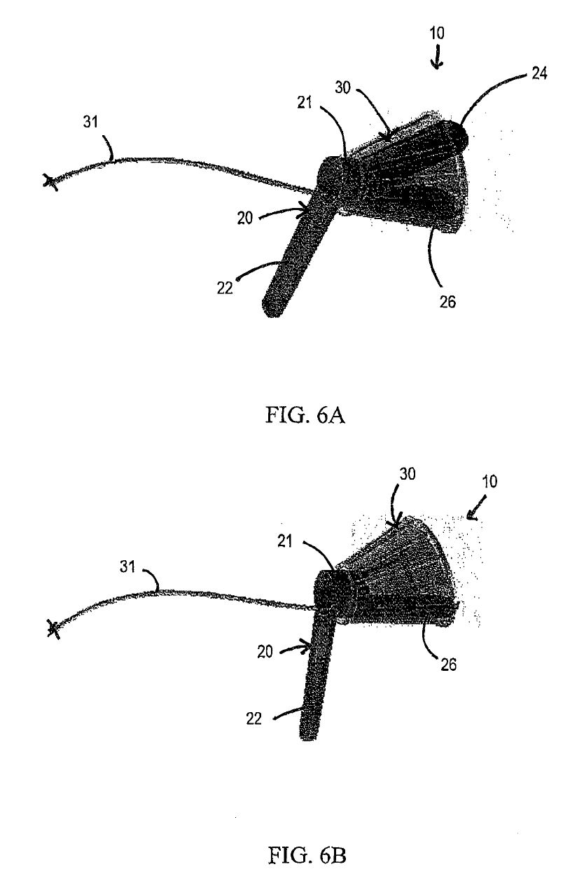

As shown in FIG. 2, an exemplary dilation system 10 comprise of speculum 20 and a sheath 30 attached thereto. As also shown in FIG. 2, sheath 30, for example, may be made of a transparent or a translucent material. An interior surface of sheath 30 is attached to the exterior surfaces of body 20 and/or jaws 24 and 26 such that sheath 30 is positioned to surround jaws 24 and 26. Sheath 30 is expandable from a constricted position with a cylindrical shape (FIG. 2) to an expanded position with a frustoconical or tapered shape (FIG. 6A). Numerous means for expanding sheath 30 are contemplated. For example, sheath 30 may be at least partially expanded when the jaw 24 is pivoted away from jaw 26, such as when moving from a closed position (FIG. 2) to an open position (FIG. 6A) under the influence of an opening force applied to jaw 24. The opening force may be applied by an elongated tool inserted into lumen 23. Alternatively, the opening force may be applied by a spring attached to jaw 24 and operated by an actuator on speculum 20, a bimetallic strip attached to jaw 24 and operated in response to the temperature of the body lumen, or any other biasing element, any of which may be mounted on body 21. In some aspects, the opening force is applied by inflating sheath 30, as described further below.

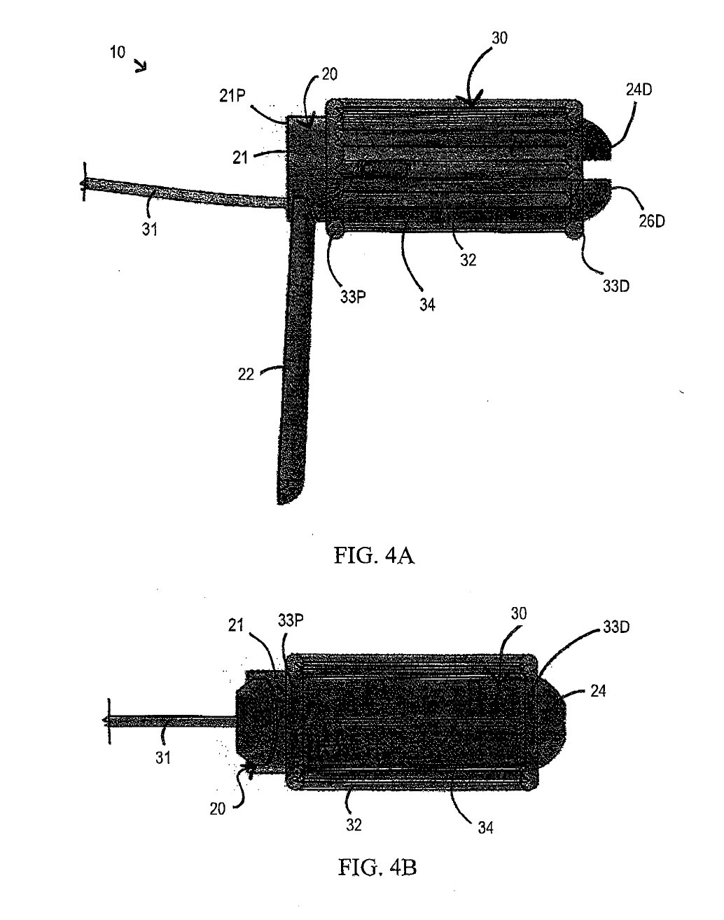

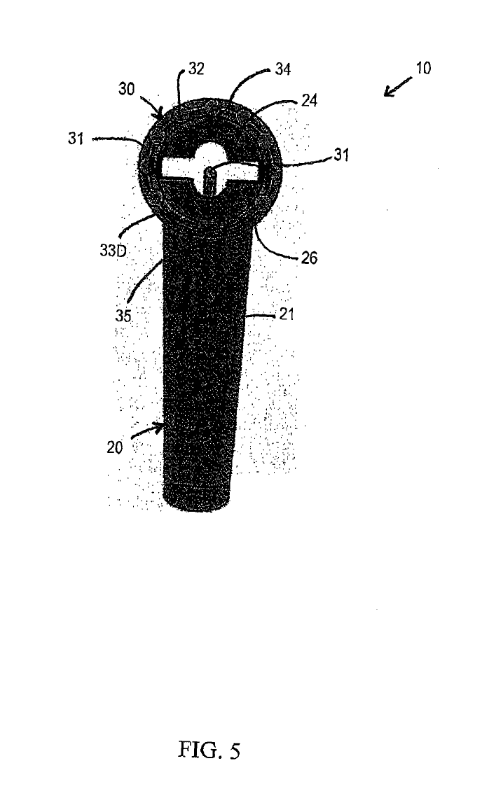

The illustrated aspect of sheath 30 has one or more resilient portions that maintain the expanded position. In FIGS. 2-6B, the one or more resilient portions comprise one or more, or a plurality, of longitudinal ribs 32 that span between a distal end of sheath 30 and a proximal end of sheath 30, each rib being configured to exert a force that maintains the expanded position. For example, each rib may house or be attached to a spring, a bimetallic strip, or like biasing element, that maintains the expanded position, similar to those described above with reference to jaw 24. Each rib 32 may also be inflatable, as described further below.

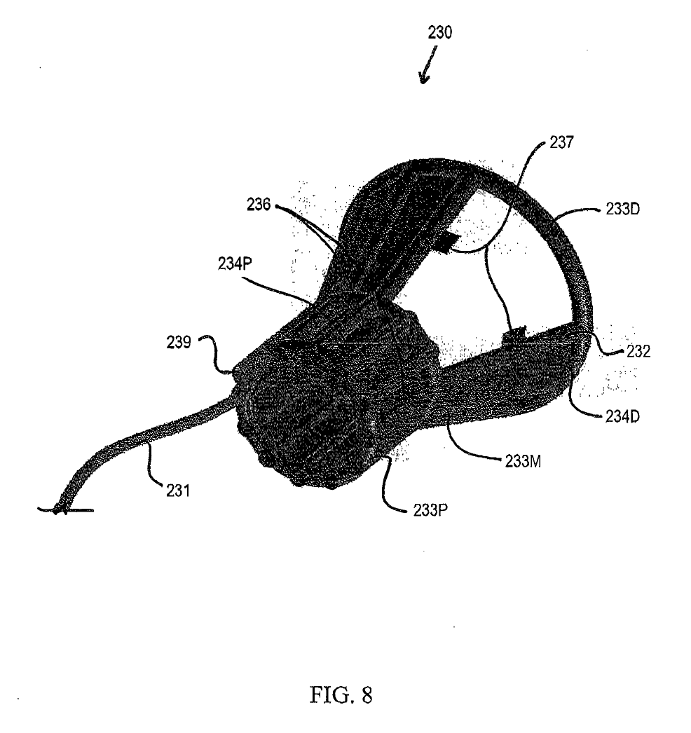

Sheath 30 of FIG. 2 further comprises at least one proximal ring 33P at the proximal end of the sheath and at least one distal ring 33D at the distal end of the sheath. Each of the proximal and distal rings 33P and 33D may also be configured to exert a force that maintains the expanded position. A biasing element may also be incorporated into rings 33P and 33D, such as those described above. Although not required, a web 34 spans between each rib 32 and/or each of the proximal and distal rings 33P and 33D. In some aspects, web 34 is removable from sheath 30, as described below with reference to FIG. 8. The entirety of sheath 30, including either or both of ribs 32 and rings 33P, 33D, may be made of a resilient biocompatible material, such as a polymeric material like silicon rubber.

Either or both of ribs 32 and rings 33P, 33D, are depicted as having a hollow cross-section. For example, each of these elements has a ring-shaped cross-sectional shape in FIGS. 2-6B. This configuration allows sheath 30 to be more readily collapsed into the constricted position and opened into the expanded position. Providing either or both of ribs 32 and rings 33P, 33D, with a hollow cross-sectional shape also defines an interior volume inside of sheath 30. For example, sheath 30 may have an interior volume defined by an interior volume of at least the one of the ribs 32. As shown in FIGS. 2-6B, the interior volume of each rib 32 may in communication with an interior volume of either or both of the proximal and distal rings 33P and 33D so as to define an contiguous interior volume of sheath 30.

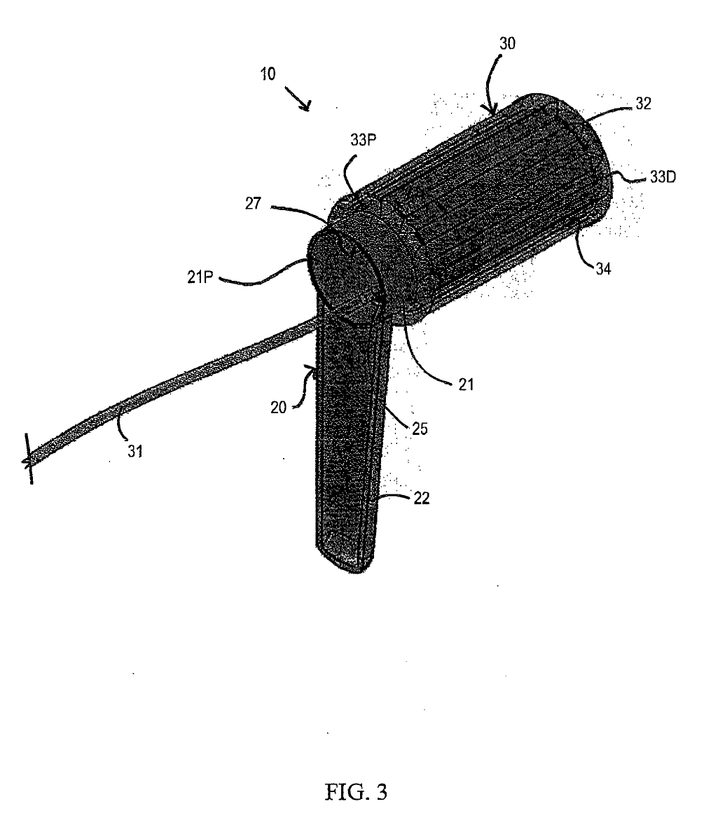

These interior volumes allow sheath 30 to be expanded by inflation when a medium is placed in the interior volume of sheath 30, resulting in an interior surface of sheath 30 that has a frustoconical or tapered shape. The medium may be placed in an interior volume of the sheath through a connection or connector valve 25 depicted as being adjacent the proximal ring 33P (FIG. 3) and connected to a source or supply line 31. In each of FIGS. 2-6B, for example, the medium is a fluid (like saline or water) or a gas (like air) that is deliver into the interior volume of sheath 30 through supply line 31. Any means of delivering the medium is contemplated, including hydraulic, pneumatic, or like means. For example, sheath 30 may be inflated with a bulbous hand pump, an automated pump, or any like source.

In some aspects, system 10 may further comprise a heating element that heats the medium as it is delivered, and/or a time-release mechanism that inflates sheath 30 at a desired rate. These features may be incorporated to maintain sheath 30 at a particular pressure and/or temperature, thereby allowing sheath 30 to dilate, for example, a vagina in a controlled manner that is responsive to the expansion or contraction of the vaginal muscles. Any known sensing and/or control technology, such as a pressure sensor, a temperature sensor, and/or a transducer may be incorporated into system 10 so as to increase the responsiveness of sheath 30.

Sheath 30 may be at least partially expanded by jaws 24 and 26, as noted above. The exterior surfaces of jaws 24 and 26 may also be attached to the interior surfaces of sheath 30, such at least jaw 24 is pivoted from a closed position (FIG. 1) to an open position (FIG. 6A) when sheath 30 is inflated. In some aspects, jaw 24 is pivoted exclusively by inflating sheath 30; while, in other aspects, jaw 24 is pivoted in part by the opening forces described above and in part by inflating sheath 30. The inflatable aspects of sheath 30 also allow jaw 24 to be omitted entirely, as in FIG. 6B, wherein the remainder of speculum 20 is provided to give sheath 30 additional rigidity, as may be required for insertion into lumen 23 and/or to maintain sheath 30 in the expanded position. According to this aspect, speculum 20 may assume any cylindrical shape suitable for providing such rigidity, including a solid, cylindrical shape that lacks a handle 22, a body lumen 23, or a jaw 24.

Speculum 20 may be removably attached to sheath 30. For example, jaws 24 and 26, as well as body 21, may be removably attached to an interior surface of sheath 30 to provide sheath 30 with the structural integrity necessary for insertion into an undilated body lumen, as noted above. Once sheath 30 is inserted and inflated into an expanded position, then either or both of jaws 24 and 26, or the entirety of speculum 20, may be removed from sheath 30 to provide additional operating space inside of lumen 23 during, for example, a cervical procedure, such as a pap smear. If a portion of web 34 is also removable, then the additional operating space may also permit use of sheath 30 during, for example, a resection procedure performed on the vaginal wall. Any of the biasing elements described above may be used to further maintain sheath 30 in the expanded position after speculum 30 or a portion of web 34 has been removed.

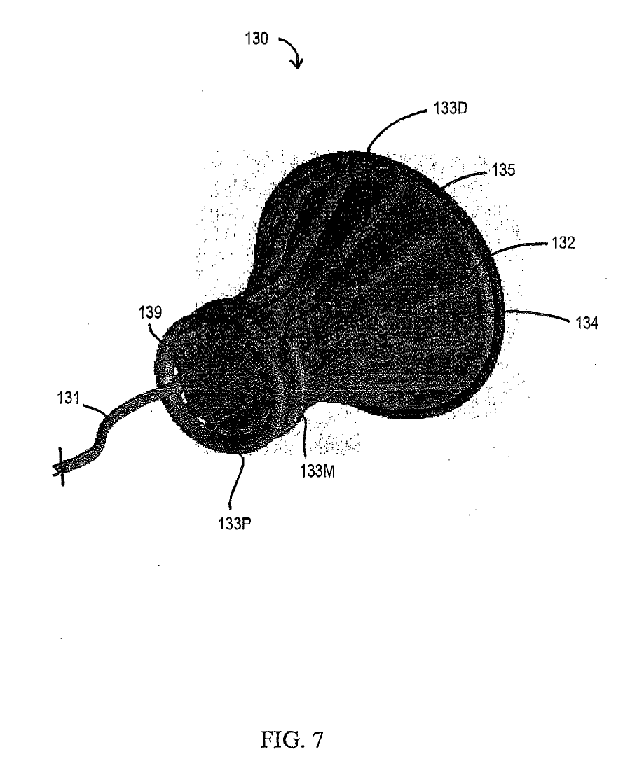

Other aspects of sheath 30 are illustrated in FIG. 7 with reference a sheath 130, and in FIG. 8 with reference to a sheath 230. Each of sheaths 130 and 230 will now be discussed in turn. Wherever possible, like element numbers have been use to describe corresponding elements of sheaths 30, 130, and 230. It should be understand that any number of combinations of these aspects are possible, each variant or iteration being part of the present disclosure.

As shown in FIG. 7, sheath 130 is a dilation device comprising of a proximal ring 133P, a medial ring 133M, and a first distal ring 133D. The medial ring 133M may be spaced apart to form an entry portion of sheath 130. For example, in some aspects, proximal ring 133P is located outside the vagina, while medial ring 133M is located inside the vagina. Rings 133P and 133M thus provide a means for locating and/or securing speculum 20 or sheath 30 in the vagina, thereby preventing those devices from moving during a procedure. Each of rings 133P, 133M, and 133D may exert an outwardly directed radial force that both maintains sheath in the expanded position shown in FIG. 7 and secures sheath 130 against the vaginal walls. Sheath 130 also has a plurality of longitudinal ribs 132 spanning between each of the proximal and distal rings 133P, 133D, and a web 134 spanning between each rib 132 and/or ring 133D, 133M, or 133P. Sheath 130, like sheath 30, is also expandable from a constricted position to an expanded position, although the shape of sheath 130 may vary from that of sheath 30. For example, first distal ring 133D may be expandable, and each rib 132 extendable, so that a first portion and a second portion of sheath 130 has a cylindrical shape in the constricted position (similar to FIG. 1), while at least the second portion has a frustoconical shape or tapered in the expanded position (as in FIG. 7). Ribs 132 of FIG. 7 have a bend that defines the transition between these first and second portions, thereby giving the second portion of sheath 130 its characteristic shape.

Sheath 130 is configured to maintain the expanded position. For example, the plurality of ribs may be configured to exert a force that maintains the expanded position. Sheath 130 is shown in FIG. 7 as having a contiguous interior volume defined by an interior volume of the plurality of ribs 132 and an interior volume of ring 133D, 133M, and 133P. Sheath 130 is expanded by inflation when a medium is placed in the contiguous interior volume through a supply line 131. In contrast to above, sheath 130 also has a second distal ring 135 attached to first distal ring 133D. Second distal ring 135 is configured to assist with rigidity and the opening of sheath 130. Ring 135 may or may not be an inflatable element of sheath 130. For example, second distal ring 135 may alternatively have a solid cross-section made of silicone or suitable polymorphic material. This configuration allows second distal ring 135 to be collapsed or folded when sheath 130 is in the constricted position, and then rapidly expanded or popped open when the body lumen is sufficiently dilated, thereby further increasing the rigidity and maintaining the expanded position of sheath 30, even if it becomes somewhat deflated during the procedure.

Sheath 130 also has a port 139. In some aspects, port 139 may be configured to guide an elongated instrument through sheath 130 and into the body lumen. For example, port 139 may be a flexible, semi-rigid, or rigid tubing used to guide a camera or a light source into the body lumen, much like port 27 described above. Port 139 may alternatively be in communication with the contiguous interior volume of sheath 130. In this aspect, port 139, in conjunction with supply line 131 and a pump operable therewith, can be used to cycle the medium through the interior volume of sheath 130. For example, the medium may be delivered from a heating element through supply line 131 at a first temperature, output from port 139 at a second temperature after a portion of heat has been absorbed by the body lumen, and then fed back into the heating element. This configuration desirably allows a vagina, for example, to be maintained a particular temperature so as to relax the vaginal muscles. The flow rate may be adjusted by a sensor to further maintain this temperature, as noted above.

Sheath 230 of FIG. 8 shares many features with sheath 130 of FIG. 7, including the rings, shapes, and capabilities described above. Sheath 230 has a medial ring 233M that separates a first portion of sheath 230 from a second portion of sheath 230. Similar to above, the first and second portions of sheath 130 have a cylindrical shape in the constricted position (similar to FIG. 1), while at least the second portion has a frustoconical or tapered shape in the expanded position (as in FIG. 7). This configuration allows sheath 230 to define a cylindrical entry portion at its proximal end. As before, rings 233P and 233M may, for example, be used to locate or secure the position of sheath 230 in a vagina.

Sheath 230 also has two sets of webs, a distal set 234D and a proximal set 234P. A portion of the distal set of webs 234D may be removably attached to sheath 230 by a set of tabs 237, while a portion of the proximal set of webs 234P is removably attached to sheath 230 by an individual tab 236. Each of tabs 236 and 237 allows one of the sets of webs 234P or 234D to be removed from sheath 230 after it has been inserted into a body lumen and expanded to, for example, permit surgical access to the vaginal wall. An exemplary set of tabs 236 or 237 may use a serrated/perforated adhesive, a Velcro® strip, a weld, a heat seal, or like means of removable attachment with webs 234P, 234D. The distal set of webs 234D, for example, may be strategically positioned to provide for a larger access opening of a fixed size, as in FIG. 8. Alternatively, one of the ribs 232 may be non-inflatable and/or even removable to permit formation of an even larger access opening. For example, one or more of ribs 232 may have an interior volume that is independent of or partitioned from the contiguous interior volume and, thus, inflatable and/or removable independent of sheath 30 so as to expand the access opening.

An exemplary dilation method 70 is also described herein with reference each aspect illustrated in FIGS. 1-8 and described above. Method 70 may comprise the step of attaching a sheath 30, for example, to a speculum 20, which, as described above, has a body 21 and at least one jaw 24 pivotally attached to body 21. Another method step 72 may comprise placing sheath 30 and speculum 20 in a body lumen, such as a vagina. Other method steps may comprise a step 74 for expanding sheath 30 from a constricted position (FIG. 2) to an expanded position (FIG. 6A); and a step 76 for maintaining the sheath in the expanded position.

Any of these exemplary method steps may be modified to accommodate a particular aspect of speculum 20 or sheath 30. For example, expanding step 74 may comprise pivoting jaw 24 from a closed position to an open position, or inflating sheath 30 by introduction of the medium, which may introduced at a particular temperature or flow rate. Sheath 30 may have an interior volume and a valve 25 in communication with the interior volume, such that expanding step 74 may further comprise attaching a supply line 31 to valve 25 and placing a medium in the interior volume through the valve to inflate sheath 30. Additional and/or alternative method steps may comprise a step 78 for detaching sheath 30 from speculum 20; and a step 80 for removing speculum 20 from the body lumen by, for example, retracting it through lumen 23 of sheath 30, or the cylindrical access port defined by sheath 230 of FIG. 7. As also described with reference to FIG. 7, sheath 230 may have at least two longitudinal ribs 132 and a web 134 removably attached to at least each of the at least two ribs 134, such that another method step 82 comprises removing the web 134 to expose an interior portion of the body lumen. At any point during this method, another step 84 may comprise performing a treatment on the interior portion of the body lumen.

While principles of the present disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the present disclosure is not to be considered as limited by the foregoing description.

We claim:

1. A dilation system, the system comprising:
a speculum with a body and at least one jaw pivotally attached to the body; and
a sheath that is attached on the body around the at least one jaw, and expandable from a constricted position to an expanded position.

2. The system of claim 1, wherein the sheath is transparent or translucent.

3. The system of claims 1, wherein the sheath is expanded when the at least one jaw is pivoted from a closed position to an open position.

4. The system of claim 1, wherein the sheath has one or more resilient portions that maintain the expanded position.

5. The system of claim 4, wherein the one or more resilient portions of the sheath are longitudinal ribs that span between a distal end of the sheath and a proximal end of the sheath, each rib being configured to exert a force that maintains the expanded position.

6. The system of claim 5, further comprising a proximal ring at the proximal end of the sheath and a distal ring at the distal end of the sheath, each of the proximal and distal rings being configured to exert a force that maintains the expanded position.

7. The system of claim 6, further comprising a web spanning between each of the one or more longitudinal ribs, the proximal ring, and the distal ring.

8. The system of claim 7, wherein the sheath has an interior volume defined by an interior volume of at least the one or more longitudinal ribs.

9. The system of claim 8, wherein the interior volume of the one or more longitudinal ribs is in communication with an interior volume of either or both of the proximal and distal rings.

10. The system of claim 9, wherein the sheath is expanded by inflation when a medium is placed in the interior volume of the sheath.

11. The system of claim 10, wherein the medium is placed in the interior volume of the sheath through a valve adjacent the proximal ring.

12. The system of claim 11, wherein the at least one jaw is attached to the sheath and pivoted from a closed position to an open position when the sheath is inflated.

13. The system of claim 12, wherein the at least one jaw is removably attached to an interior surface of the sheath, and the at least one jaw is removable from sheath when the sheath is inflated.

14. A dilation device, the device comprising:
at least one proximal ring spaced apart from at least one distal ring;
a plurality of longitudinal ribs spanning between each of the proximal and distal rings; and
a web spanning between at least each of the plurality of ribs,
wherein the sheath is expandable from a constricted position to an expanded position.

15. The device of claim 14, wherein the at least one distal ring is expandable so that the sheath has a cylindrical shape in the constricted position and a frustoconical shape in the expanded position.

16. The device of claim 15, wherein at least the plurality of ribs are configured to maintain the expanded position.

17. The device of claim 16, wherein the sheath has a contiguous interior volume defined by an interior volume of the plurality of ribs, an interior volume of the at least one proximal ring, and an interior volume of the at least one distal ring.

18. The system of claim 17, wherein the sheath is expanded by inflation when a medium is placed in the contiguous interior volume.

19. The system of claim 18, wherein at least a portion of the web is removably attached to at least the plurality of ribs.

20. A dilation method, the method comprising:
attaching a sheath to a speculum, the speculum including a body and at least one jaw pivotally attached to the body;
placing the sheath and the speculum into a body lumen;
expanding the sheath from a constricted position to an expanded position; and
maintaining the sheath in the expanded position.

21. The method of claim 20, wherein the expanding step comprises pivoting the at least one jaw from a closed position to an open position.

22. The method of claim 21, further comprising:
detaching the sheath from the speculum; and
removing the speculum from the body lumen.

23. The method of claim 20, wherein the expanding step comprises inflating the sheath.

24. The method of claim 23, wherein the sheath has an interior volume and a valve in communication with the interior volume, further comprising placing a medium in the interior volume through the valve to inflate the sheath.

25. The method of claim 24, further comprising controlling a temperature or a pressure of the medium.

26. The method of claim 20, wherein the sheath has at least two longitudinal ribs and a Web removably attached to each of the at least two ribs, further comprising removing the web to expose an interior portion of the body lumen.

27. The method of claim 26, further comprising performing a treatment on the interior portion of the body lumen.
